(19) United States
(12) Patent Application Publication
(10) Pub. No.: US 2025/0154016 A1
(43) Pub. Date: May 15, 2025

(54) AMMONIA SYNTHESIS AND UREA SYNTHESIS WITH REDUCED CO2 FOOTPRINT

(71) Applicants: thyssenkrupp Uhde GmbH, Dortmund (DE); thyssenkrupp AG, Essen (DE)

(72) Inventors: Frederick KESSLER, Menden (DE); Yevgeny MAKHYNYA, Muelheim an der Ruhr (DE)

(73) Assignees: thyssenkrupp Uhde GmbH, Dortmund (DE); thyssenkrupp AG, Essen (DE)

(21) Appl. No.: 18/729,832

(22) PCT Filed: Jan. 19, 2023
(86) PCT No.: PCT/EP2023/051284

§ 371 Date: Jul. 17, 2024

(30) Foreign Application Priority Data
Jan. 19, 2022 (BE) BE 2022/5030
Jan. 19, 2022 (DE) 10 2022 200 572.4

(51) Int. Cl. C01C 1/04; C01B 3/02; C07C 273/04

(52) U.S. Cl. CPC C01C 1/0488; C01B 3/025; C01C 1/0464; C07C 273/04; C01B 2203/0244; C01B 2203/0415; C01B 2203/0475; C01B 2203/0811

(57) ABSTRACT

The present invention relates to a plant for the synthesis of ammonia, wherein the plant includes at least one reformer for converting a hydrocarbon into hydrogen, wherein the plant includes a converter for converting hydrogen and nitrogen into ammonia, wherein the converter is integrated into a recirculation loop, wherein a first carbon dioxide separator is arranged between the reformer and the recirculation loop, wherein the recirculation loop includes an ammonia separator.

The invention relates to a plant complex and to a process for producing ammonia from a combination of hydrogen from natural gas and from electrolysis utilizing renewable energies and concomitant use of carbon dioxide in urea synthesis and/or of nitrogen in ammonia synthesis, which are formed in the production of hydrogen from natural gas.

Hydrogen is produced in particular by steam reforming, in which a hydrocarbon is reacted with steam to form carbon monoxide and hydrogen, with subsequent conversion of the carbon monoxide into carbon dioxide and hydrogen in a water-gas shift reaction. This endothermic reaction requires an external supply of energy, obtained for example by burning hydrocarbons in an adjoining combustion chamber. An alternative is to use autothermal reforming, in which a partial oxidation that provides the necessary energy takes place.

Normally, methane is reacted with water and air in the primary and secondary reformers to form carbon dioxide and hydrogen, normally with establishment of the target composition of 3:1 hydrogen to nitrogen. This normally takes place in steps, wherein, in a primary reformer, methane is first reacted with water with an input of energy and then, in a secondary reformer, with an input of oxygen, usually in the form of air, this being followed by a shift reaction in which carbon monoxide reacts with water to form carbon dioxide and hydrogen. Thus, after removal of the carbon dioxide and normally the conversion of any contained carbon monoxide present into methane, this mixture can be converted directly into ammonia in a converter.

After the synthesis, the ammonia is often converted directly into urea with carbon dioxide. This is often done using the carbon dioxide from the reformer, i.e. from the hydrogen production process.

WO 2019/110 443 A1 discloses a process for providing CO₂ for urea synthesis from flue gas and synthesis gas.

EP 3 390 354 B1 discloses a process for providing carbon dioxide for the synthesis of urea.

Likewise known is the generation of hydrogen by electrolysis, in particular from renewable energy sources and thus in a CO₂-free manner. There is therefore of course interest in using this so-called “green” hydrogen also for the synthesis of ammonia, so as to obtain “green” ammonia. Since no nitrogen is generated during the production of hydrogen, this is normally obtained from air separation, which is however an energy-intensive process.

In order to operate the process within the primary reformer, a primary reformer has a burner side in which a fuel gas, usually natural gas, is burned in air, thereby providing the necessary thermal energy. The flue gas exiting the burner side mainly comprises nitrogen and carbon dioxide, two substances that, although actually usable within the plant, are usually released directly into the environment.

The object of the invention is to use the flue gas generated on the burner side of the primary reformer at least partially within the process and thus to save energy in the overall process and/or to reduce emissions.

This object is achieved by plants having the features specified in claim 1 and by the processes having the features specified in claim 15 and claim 16. Advantageous developments will be apparent from the dependent claims, the description that follows, and the drawings.

The plant is used for the synthesis of ammonia and optionally for the further synthesis of urea from the ammonia produced. Such combined plants for the production of nitrogen-containing fertilizer are known and customary. These plants may also include other components, for example a nitric acid plant for producing nitric acid from ammonia and especially an apparatus downstream thereof for producing ammonium nitrate as fertilizer from ammonia and nitric acid. The plant includes at least one reformer for converting a hydrocarbon into hydrogen. Normally, the plant includes one reformer. For example, the reformer includes a primary reformer and a secondary reformer for converting a hydrocarbon into hydrogen; in particular, steam reforming is used here in which, in a first step, methane in particular is reacted with steam and in a second step with air, wherein a downstream water-gas shift reaction normally takes place in which the carbon monoxide produced is reacted with steam to form carbon dioxide and hydrogen. Alternatively, the reformer may be an autothermal reformer in which hydrocarbon, steam, and oxygen are brought together in such a way that the energy required for conversion into hydrogen is generated directly from the burning. In contrast to steam reforming, there is no need for any external supply of energy here. The plant also includes a converter for converting hydrogen and nitrogen into ammonia. The converter is furnished with a catalyst and is operated at high pressure and high temperature. Since the conversion reaction is an equilibrium reaction in which near-complete conversion does not occur, the synthesis gas is conveyed in a recirculation loop so as to be able to return unreacted reactants to the converter. The process is known as the Haber process. The converter is accordingly integrated into a recirculation loop. Arranged between the reformer and the recirculation loop is a first carbon dioxide separator. This separator separates the carbon dioxide formed from the starting material, in particular methane, in order to then supply this to, for example and in particular, a urea synthesis apparatus. Thus, downstream of the first carbon dioxide separator, a gas stream containing nitrogen and hydrogen in a ratio of 1:3 and free of other components (except perhaps in trace amounts) is provided for the synthesis of ammonia. Between the first carbon dioxide separator and the recirculation loop there is usually a methanator, an apparatus for converting any traces of carbon monoxide and carbon dioxide to methane, so as to prevent poisoning of the catalyst. The recirculation loop includes an ammonia separator. This separator separates the ammonia product from the unreacted reactant stream of nitrogen and hydrogen. In addition, the recirculation loop normally includes heat exchangers between the converter and the ammonia separator for cooling and between the ammonia separator and the converter for heating. In addition, the recirculation loop normally includes a compressor.

According to the invention, the plant includes a further hydrogen source. The further hydrogen source is preferably a water electrolysis. The water electrolysis is preferably operated using renewable energies. The hydrogen produced in this way is accordingly free of carbon dioxide emissions and is thus considered to be “green” hydrogen. The further hydrogen source is connected to the recirculation loop in such a way that hydrogen is supplied to the recirculation loop. For this, the hydrogen is preferably first mixed with nitrogen and then compressed by one or more compressors. The plant includes a combustion apparatus. For example, the combustion apparatus may be the burner side of a primary reformer. Alternatively, the combustion apparatus may be a steam generation apparatus. A steam generation apparatus is for example used to operate the compressors. Similarly, the exhaust gases from two or more combustion apparatuses may also be combined if a larger gas stream is desired. The combustion apparatus, for example the burner side of the primary reformer, is connected to a second carbon dioxide separator. The second carbon dioxide separator is connected to the recirculation loop in such a way that nitrogen is supplied to the recirculation loop. Preferably, the hydrogen from the further hydrogen source and the nitrogen from the second carbon dioxide separator are first combined and compressed together via one or more compressors. The advantage is that the combustion on the burner side makes it possible to provide nitrogen in further process steps relatively easily through a separation of carbon dioxide, allowing a hydrogen to nitrogen ratio of 3:1 to be established without an energy-intensive air separation.

In a further embodiment of the invention, the reformer includes a primary reformer and a secondary reformer for converting a hydrocarbon into hydrogen. The primary reformer has a hydrogen side and a burner side. On the hydrogen side, hydrocarbon is reacted with steam to form carbon monoxide or carbon dioxide and hydrogen. The necessary energy is provided by combustion, in particular of hydrocarbon with oxygen, in particular with air. The burner side is here the combustion apparatus, in which hydrocarbon is burned with air in the burner side of the primary reformer. The burner side of the primary reformer is connected to a second carbon dioxide separator.

In a further embodiment of the invention, the combustion apparatus is a steam generation apparatus.

In a further embodiment of the invention, the reformer is an autothermal reformer.

In a further embodiment of the invention, the plant further includes a urea synthesis apparatus for the synthesis of urea from ammonia and carbon dioxide. For the separated carbon dioxide, the first carbon dioxide separator is connected to the urea synthesis apparatus. Normally, the amount of carbon dioxide separated is slightly lower than the amount of ammonia produced from the nitrogen and hydrogen, with the result that the ammonia is incompletely converted into urea. The ammonia separator is connected to the urea synthesis apparatus in an ammonia conducting manner. Here, an intermediate storage tank may in addition be arranged in the ammonia conducting connection.

In a further embodiment of the invention, the second carbon dioxide separator is an ammonia-water scrubber. Such scrubbers are known for example from WO 2019/110 443 A1 or EP 3 390 354 B1.

In a further embodiment of the invention, the nitrogen from the second carbon dioxide separator is supplied to the recirculation loop such that the nitrogen from the second carbon dioxide separator is introduced into the secondary reformer. This results in reaction of any residual oxygen still present in the secondary reformer. It is not necessary here for the entire nitrogen stream to be conveyed to the secondary reformer. Rather, this stream can be adjusted in line with the amount of additional hydrogen.

In a further embodiment of the invention, an apparatus for removing oxygen is arranged between the second carbon dioxide separator and the recirculation loop. In this case, an additional compressor is preferably provided to allow equalization of the pressure with the high level in the recirculation loop.

In a further embodiment of the invention, the burner side of the primary reformer is operated with a substoichiometric amount of oxygen or an excess of methane. This is unusual for the actual operation as the burner side, but ensures that the oxygen is completely consumed.

In a further embodiment of the invention, the second carbon dioxide separator is connected to the recirculation loop in such a way that nitrogen is supplied to the recirculation loop via the autothermal reformer. This too allows the residual oxygen present to be reliably reacted.

In a further embodiment of the invention, a dust extraction apparatus (dedusting apparatus) is arranged between the burner side of the primary reformer and the second carbon dioxide separator. Preferably, a desulfurization apparatus and/or a denitrification apparatus may additionally be arranged downstream of the dust extraction apparatus.

In a further embodiment of the invention, the further hydrogen source and the second carbon dioxide separator are connected to the recirculation loop in such a way that the hydrogen stream from the further hydrogen source is first combined with the nitrogen stream from the second carbon dioxide separator, then conveyed through a first compressor and thereafter conveyed through a methanator and then supplied to the recirculation loop. This makes it possible in particular for the capacity of the ammonia synthesis to be more easily increased, since the existing synthesis gas production in the reformer remains unchanged and is thus augmented by the additional gas stream directly upstream of the converter.

In a further embodiment of the invention, the combustion apparatus, for example the burner side of the primary reformer, is connected to the secondary reformer. As a result, not just nitrogen but also carbon dioxide and residual oxygen are supplied to the gas stream for the production of hydrogen. The remaining residual oxygen is reacted in the secondary reformer. Since carbon dioxide separation takes place downstream of the secondary reformer, the carbon dioxide produced on the burner side can thus in that case also be separated in the same step.

In a further embodiment of the invention, a dust extraction apparatus is arranged between the combustion apparatus, for example and preferably the burner side of the primary reformer, and the reformer, for example and preferably the secondary reformer. Preferably, a desulfurization apparatus and/or a denitrification apparatus may additionally be arranged downstream of the dust extraction apparatus.

In a further embodiment of the invention, a compressor is arranged between the combustion apparatus, preferably the burner side of the primary reformer, and the reformer, preferably the secondary reformer.

In a further embodiment of the invention, the plant is used for the further synthesis of urea from the ammonia produced. Such combined plants for the production of nitrogen-containing fertilizer are known and customary. These plants may also include other components, for example a nitric acid plant for producing nitric acid from ammonia and especially an apparatus downstream thereof for producing ammonium nitrate as fertilizer from ammonia and nitric acid. The plant further includes a urea synthesis apparatus for the synthesis of urea from ammonia and carbon dioxide. The ammonia separator is connected to the urea synthesis apparatus in an ammonia conducting manner. Here, an intermediate storage tank may in addition be arranged in the ammonia conducting connection. The second carbon dioxide separator is connected to the urea synthesis apparatus in such a way that carbon dioxide is supplied to the urea synthesis apparatus.

In a further embodiment of the invention the plant includes a further hydrogen source. The further hydrogen source is connected to the recirculation loop in such a way that hydrogen is supplied to the recirculation loop. The burner side of the primary reformer is connected to a second carbon dioxide separator. The second carbon dioxide separator is connected to the recirculation loop in such a way that nitrogen is supplied to the recirculation loop. The burner side of the primary reformer is connected to the secondary reformer.

In a further embodiment of the invention the plant includes a further hydrogen source. The further hydrogen source is connected to the recirculation loop in such a way that hydrogen is supplied to the recirculation loop. The burner side of the primary reformer is connected to a second carbon dioxide separator. The second carbon dioxide separator is connected to the recirculation loop in such a way that nitrogen is supplied to the recirculation loop. In addition, the second carbon dioxide separator is connected to the urea synthesis apparatus in such a way that carbon dioxide is supplied to the urea synthesis apparatus. This makes it possible to achieve optimal use of all gas streams. Through the additional, in particular “green”, hydrogen produced it is possible to achieve a ratio of nitrogen to hydrogen to carbon dioxide of, for example, 2:6:1.

In a further embodiment of the invention the plant includes a further hydrogen source. The further hydrogen source is connected to the recirculation loop in such a way that hydrogen is supplied to the recirculation loop. The burner side of the primary reformer is connected to the secondary reformer. The burner side of the primary reformer is further connected to a second carbon dioxide separator, and the second carbon dioxide separator is connected to the urea synthesis apparatus in such a way that carbon dioxide is supplied to the urea synthesis apparatus.

In this embodiment, all gas streams must always be conveyed completely in accordance with the interconnection. For example, in the various embodiments it is also possible for substreams, in particular the exhaust gas of the burner side, the nitrogen stream or the carbon dioxide stream of the second carbon dioxide separator, to be removed and discarded or otherwise used.

In a further aspect, the invention relates to a process for expanding the capacity of an existing plant according to the prior art. In this case, the plant is expanded to include a further hydrogen source. The water electrolysis is preferably operated using renewable energies. The hydrogen produced in this way is accordingly free of carbon dioxide emissions and is thus considered to be “green” hydrogen. The further hydrogen source is in this case connected to the recirculation loop in such a way that hydrogen is supplied to the recirculation loop. This allows the capacity of the ammonia synthesis to be increased. The burner side of the primary reformer is connected to the secondary reformer. As a result, both nitrogen and carbon dioxide are supplied to the synthesis. The carbon dioxide is separated together with the carbon dioxide produced in the reformer and supplied to the urea synthesis. Not only does this allow the total capacity to be expanded, but the carbon dioxide footprint is reduced too.

In a further aspect, the invention relates to a further process for expanding the capacity of an existing plant according to the prior art. The plant is expanded to include a further hydrogen source and a second carbon dioxide separator. The water electrolysis is preferably operated using renewable energies. The hydrogen produced in this way is accordingly free of carbon dioxide emissions and is thus considered to be “green” hydrogen. The further hydrogen source is connected to the recirculation loop in such a way that hydrogen is supplied to the recirculation loop. This increases the amount of hydrogen supplied to the converter. The burner side of the primary reformer is connected to the second carbon dioxide separator. In addition, the second carbon dioxide separator is connected to the recirculation loop in such a way that nitrogen is supplied to the recirculation loop. Thus, in addition to additional hydrogen, nitrogen is also supplied, thereby increasing the overall capacity. In addition, the second carbon dioxide separator is connected to the urea synthesis apparatus in such a way that carbon dioxide is supplied to the urea synthesis apparatus. This ensures that the increased amount of ammonia is reflected in increased production of urea.

The plant of the invention is more particularly elucidated hereinbelow with reference to exemplary embodiments depicted in the drawings.

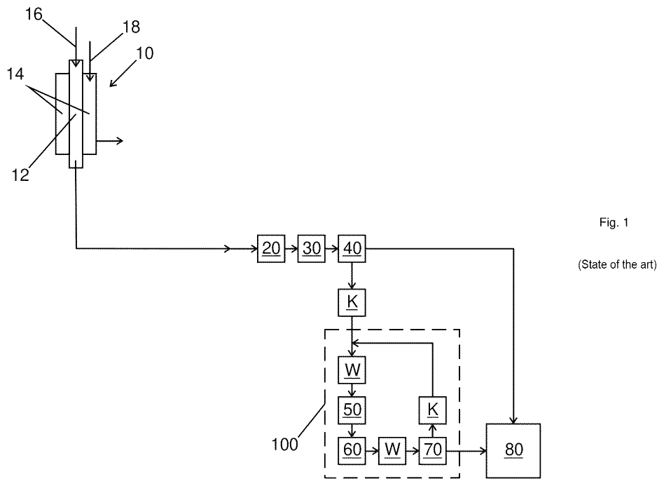

FIG. 1 State of the art

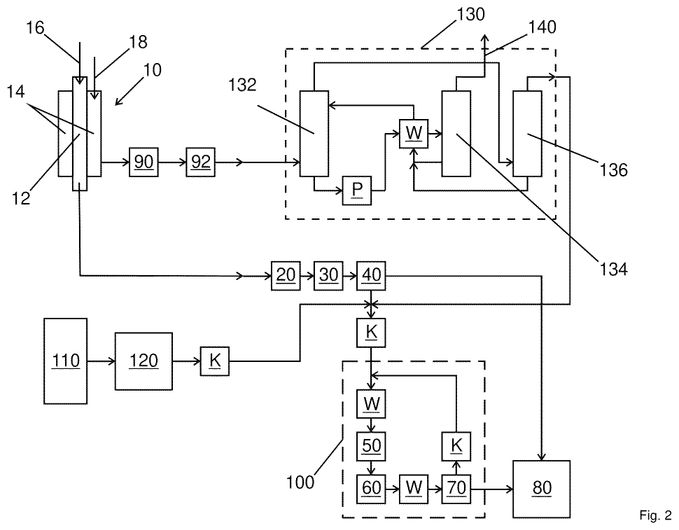

FIG. 2 First exemplary embodiment

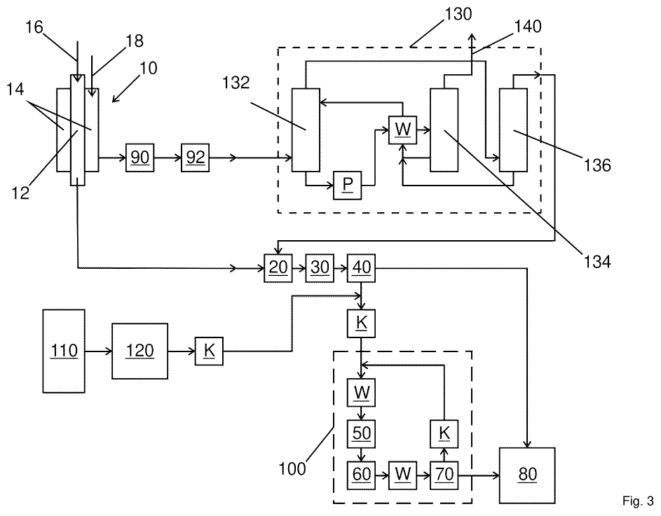

FIG. 3 Second exemplary embodiment

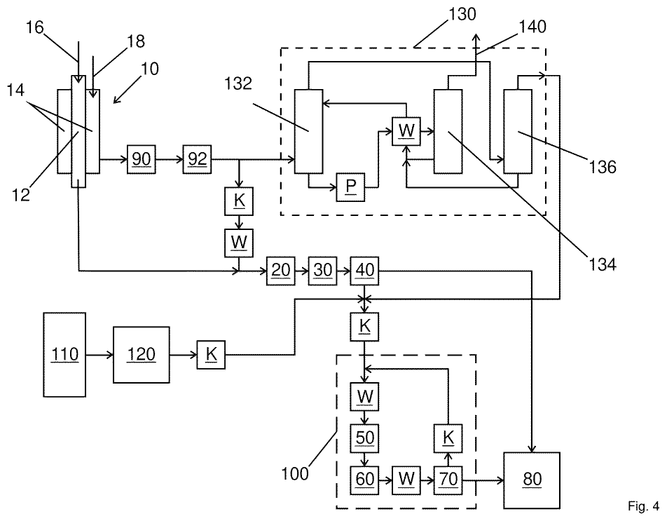

FIG. 4 Fifth exemplary embodiment

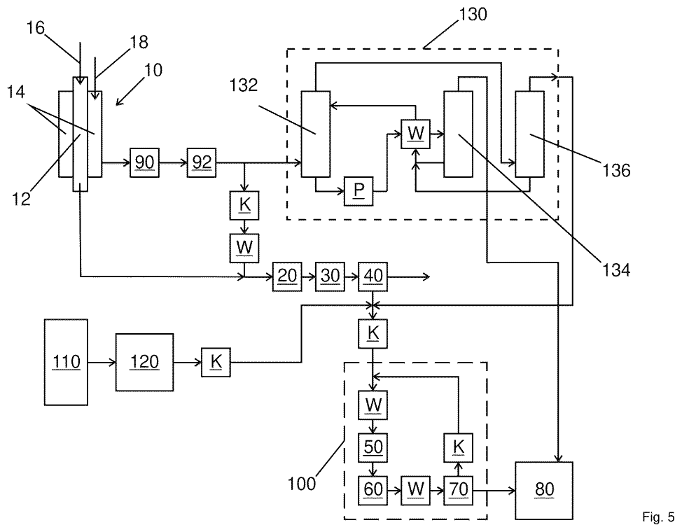

FIG. 5 Sixth exemplary embodiment

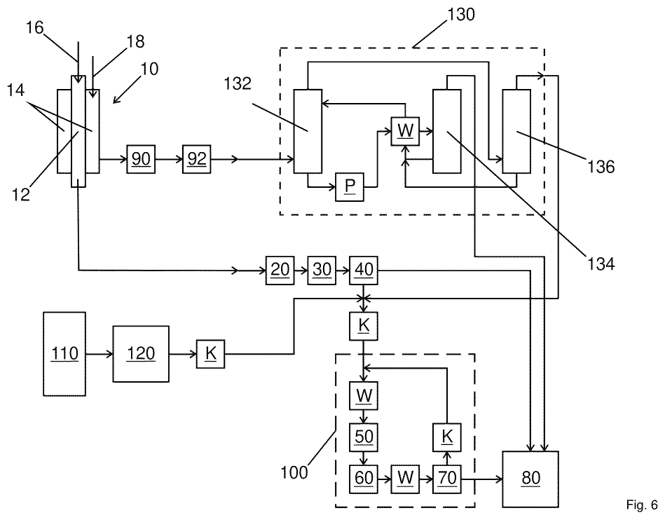

FIG. 6 Seventh exemplary embodiment

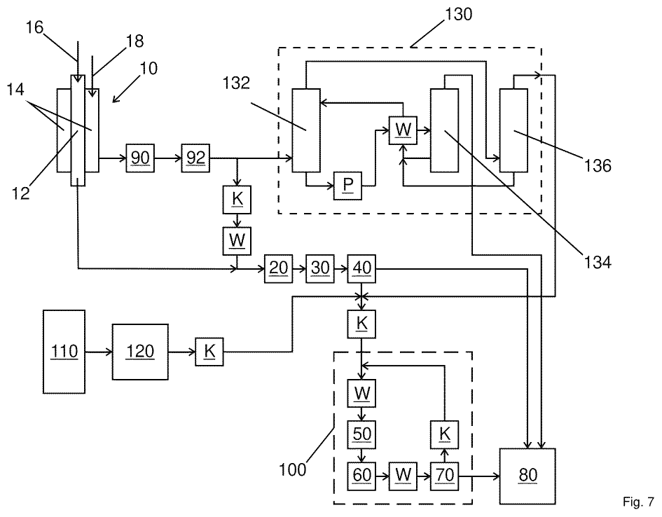

FIG. 7 Ninth exemplary embodiment

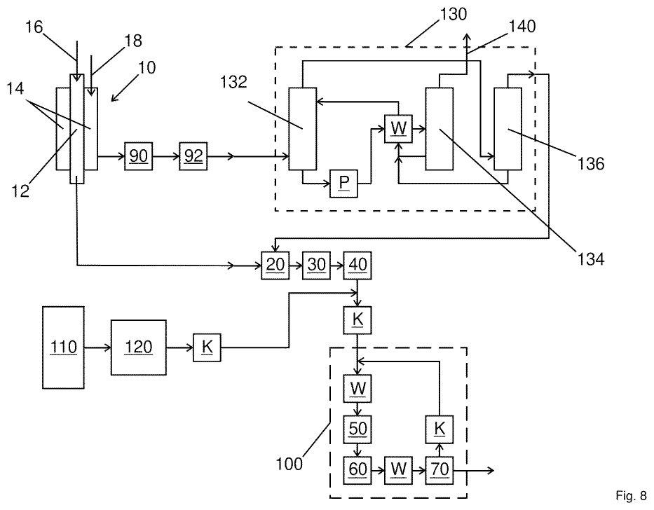

FIG. 8 Tenth exemplary embodiment

Firstly, the components common to all exemplary embodiments are discussed, as illustrated by the state of the art in FIG. 1; thereafter, only the additional components in each case are discussed.

These representations are simplified and are schematic only. For example, compressors K may also be multistage. Also normally present is an apparatus known as a methanator, which is arranged upstream of the supply to the recirculation loop 100 and converts residual traces of carbon dioxide and carbon monoxide, which are catalyst poisons, into methane. Such variants, which are common in ammonia synthesis, are omitted here for simplicity. Likewise, the two compressors, which are arranged downstream of the first carbon dioxide separator 40 and the ammonia separator 70, may be identical. Such variants and arrangements for gas conveyance are known to those skilled in the art and have no direct influence on the invention.

The plant according to the state of the art shown in FIG. 1 is used for the synthesis of ammonia with further reaction to urea, wherein the hydrogen is produced by steam reforming and ammonia via the Haber process.

In a primary reformer 10, methane and steam are supplied as a hydrogen source 16 on the hydrogen side 12. The energy necessary for the reaction is generated and provided by a combustion on the burner side 14. For example, a mixture of methane and air is provided via the fuel gas supply 18. Ideally, a gas mixture of nitrogen and carbon dioxide is thus produced on the burner side 14. In reality, about 2% by volume of oxygen may be present as an additional component. The gas mixture produced on the hydrogen side 12 is conveyed into a secondary reformer 20, where air is normally added. Here, methane, for example, is reacted with oxygen to form carbon monoxide and hydrogen. In a subsequent shift reactor 30, which normally consists of two separate reactors at different temperatures, carbon monoxide is reacted with water to form carbon dioxide and hydrogen. The carbon dioxide is then separated in a first carbon dioxide separator 40. The gas, which should then contain only nitrogen and hydrogen, is conveyed via a compressor K into the recirculation loop 100. In the recirculation loop 100, the gas is first heated in a heat exchanger W and then supplied to the converter 50. The heat of reaction evolved during the reaction is then dissipated in a cooler 60. The gas stream is then further cooled in a heat exchanger W, with the result that ammonia is separated in the ammonia separator 70. Unreacted hydrogen and unreacted nitrogen remain in the gas stream. These gases are recycled by a compressor, giving rise to the recirculation loop 100. The ammonia separated in the ammonia separator 70 and the carbon dioxide separated in the first carbon dioxide separator are reacted to form urea and water in the urea synthesis apparatus 80. This is normally followed by the performance of a granulation, with or without further additives, in order for the urea to be sold as a fertilizer.

The exemplary embodiments will now be presented hereinbelow with reference to the additional components and connections.

FIG. 2 shows a first exemplary embodiment. In this embodiment, a further hydrogen source is present. This consists solely by way of example of a solar and wind farm 110. Here, electricity is generated from renewable sun and wind energy sources. This electricity is used to produce hydrogen in the water electrolysis 120. The hydrogen may be stored temporarily in a storage tank to compensate for fluctuations in solar radiation and wind. Likewise, to smooth the supply, a battery may correspondingly be present between the solar and wind farm 110 and the water electrolysis 120. The (“green”) hydrogen thus produced is combined with the gas stream exiting the reformer and supplied to the recirculation loop 100. However, this results in nitrogen being present in a substoichiometric amount. In order not to have to operate an energy-intensive air separation, the nitrogen is extracted from the exhaust gas of the burner side 14 of the primary reformer 10. For this purpose, the gas first undergoes dust extraction in a dust extraction apparatus 90. Optionally, the gas can then be conveyed through a desulfurization apparatus 92, especially in regions in which sulfur-containing natural gas is used. The gas is then conveyed into the second carbon dioxide separator 130, which is designed as an ammonia-water scrubber, such as those shown for example in WO 2019/110 443 A1 or EP 3 390 354 B1. The second carbon dioxide separator 130 includes a CO₂ dissolution apparatus 132 in which the carbon dioxide is dissolved in ammonia water. The solution is then compressed via a pump P, for example, to 150 bar, and conveyed via a heat exchanger W into the CO₂ release apparatus 134. There, the carbon dioxide is released again at elevated temperatures and can be released via CO₂ discharge 140. In the simplest case, it is released into the environment. However, it can also be stored or reacted in order to avoid CO₂ emissions. The ammonia water is conveyed from the CO₂ release apparatus 134 via the heat exchanger W back into the CO₂ dissolution apparatus 132. The second carbon dioxide separator 130 additionally includes an ammonia capture scrubber 136. This affords a pure nitrogen stream that is then supplied to the gas stream supplied to the recirculation loop 100. In order to obtain the correct stoichiometry, it is also possible for the nitrogen gas stream to be supplied here only in part. Excess nitrogen can for example be simply released into the environment or used as an inert gas in further syntheses. Since oxygen and nitrogen are similar than nitrogen and carbon dioxide, separation from this gas stream of the burner side 14 is more efficient than air separation.

FIG. 3 shows a second exemplary embodiment. The differs from the first exemplary embodiment in that the nitrogen stream from the second carbon dioxide separator 130 is conveyed into the secondary reformer 20 in order to burn residual oxygen there.

FIG. 4 showed a fifth exemplary embodiment. In this embodiment, a further hydrogen source is present. This consists solely by way of example of a solar and wind farm 110. Here, electricity is generated from renewable sun and wind energy sources. This electricity is used to produce hydrogen in the water electrolysis 120. The hydrogen may be stored temporarily in a storage tank to compensate for fluctuations in solar radiation and wind. Likewise, to smooth the supply, a battery may correspondingly be present between the solar and wind farm 110 and the water electrolysis 120. The (“green”) hydrogen thus produced is combined with the gas stream exiting the reformer and supplied to the recirculation loop 100. However, this results in nitrogen being present in a substoichiometric amount. In order not to have to operate an energy-intensive air separation, the nitrogen is extracted from the exhaust gas of the burner side 14 of the primary reformer 10. For this purpose, the gas first undergoes dust extraction in a dust extraction apparatus 90. Optionally, the gas can then be conveyed through a desulfurization apparatus 92, especially in regions in which sulfur-containing natural gas is used. The gas is then supplied to the secondary reformer 20 via a compressor K and a heat exchanger W. The sequence of the compressor K and heat exchanger may also be reversed. This firstly rebalances the ratio of hydrogen to nitrogen. In addition, more carbon dioxide is introduced, which is separated in the first carbon dioxide separator 40 and supplied to the urea synthesis apparatus 80. This makes it very easy to increase the total amount of urea produced and at the same time reduce the CO₂ footprint. The advantage of this fifth exemplary embodiment is the flexible conveyance, whereby one part of the carbon dioxide is supplied from the burner side 14 to the urea synthesis apparatus 80 and another part released via the CO₂ discharge 140, in order that the correct stoichiometry can thus be easily established. The sequence of the compressor K and heat exchanger may also be reversed.

FIG. 5 showed a sixth exemplary embodiment, which differs from the fifth embodiment in that the carbon dioxide from the second carbon dioxide separator 130 is used in the urea synthesis apparatus 80. In this embodiment, the carbon dioxide generated in the first carbon dioxide separator 40 is discarded, because this is at a lower pressure level.

FIG. 6 showed a seventh exemplary embodiment. In many plants, less carbon dioxide will be provided from the first carbon dioxide separator 40 than would be necessary for the complete conversion of ammonia into urea. In order to increase production, another source of carbon dioxide must therefore be found. This can be found in the exhaust gas of the burner side 14 of the primary reformer 10. For this purpose, the gas first undergoes dust extraction in a dust extraction apparatus 90. Optionally, the gas can then be conveyed through a desulfurization apparatus 92, especially in regions in which sulfur-containing natural gas is used. The gas is then conveyed into the second carbon dioxide separator 130, which is designed as an ammonia-water scrubber, such as those shown for example in WO 2019/110 443 A1 or EP 3 390 354 B1. The second carbon dioxide separator 130 includes a CO₂ dissolution apparatus 132 in which the carbon dioxide is dissolved in ammonia water. The solution is then compressed via a pump P, for example, to 150 bar, and conveyed via a heat exchanger W into the CO₂ release apparatus 134. There, at elevated temperatures, the carbon dioxide is released again and is then supplied to the urea synthesis apparatus 80, wherein the high pressure of the CO₂ release apparatus 134 provides the carbon dioxide at the correct pressure level. The ammonia water is conveyed from the CO₂ release apparatus 134 via the heat exchanger W back into the CO₂ dissolution apparatus 132. In addition, the second carbon dioxide separator 130 includes an ammonia capture scrubber 136, as a result of which no ammonia is discharged into the environment with the nitrogen via the nitrogen discharge 150 or is introduced with the nitrogen as inert gas in further syntheses. The advantage of this seventh exemplary embodiment is that not only is the nitrogen stream supplied to the recirculation loop 100, and thus to the ammonia synthesis, the carbon dioxide stream is supplied to the urea synthesis apparatus 80 too. This fifth embodiment is particularly preferable in a retrofit, since only the second carbon dioxide separator 130 and a further hydrogen source will be provided, thus making it possible to achieve increased conversion in the amount produced while simultaneously reducing the CO₂ footprint.

FIG. 7 showed a ninth exemplary embodiment, which represents a combination of the first exemplary embodiment, the third exemplary embodiment, and the fourth exemplary embodiment. During operation of the plant this means that all options permitting different operating modes are available, for example in order to be able to adapt to fluctuations in the amounts of energy generated from renewable sources.

The tenth exemplary embodiment shown in FIG. 8 differs from the second exemplary embodiment shown in FIG. 3 in that it does not include a urea synthesis apparatus 80. This embodiment is particularly suitable for expanding the capacity of an existing plant for the synthesis of ammonia.

### LIST OF REFERENCE NUMERALS

10 Primary reformer
12 Hydrogen side
14 Burner side
16 Hydrogen source
18 Fuel gas supply
20 Secondary reformer
30 Shift reactor
40 First carbon dioxide separator
50 Converter
60 Cooler
70 Ammonia separator
80 Urea synthesis apparatus
90 Dust extraction apparatus
92 Desulfurization apparatus
100 Recirculation loop
110 Solar and wind farm
120 Water electrolysis
130 Second carbon dioxide separator
132 CO₂ dissolution apparatus
134 CO₂ release apparatus
136 Ammonia capture scrubber
140 CO₂ discharge
150 Nitrogen discharge
K Compressor
P Pump
W Heat exchanger

1-16. (canceled)

17. A plant for synthesis of ammonia, comprising:
a reformer for converting a hydrocarbon into hydrogen;
a converter for converting hydrogen and nitrogen into ammonia, wherein the converter is integrated into a recirculation loop, wherein a first carbon dioxide separator is arranged between the reformer and the recirculation loop, and wherein the recirculation loop includes an ammonia separator;
a further hydrogen source, wherein the further hydrogen source is connected to the recirculation loop in such a way that hydrogen is supplied to the recirculation loop; and
a combustion apparatus, wherein the combustion apparatus is connected to a second carbon dioxide separator, wherein the second carbon dioxide separator is connected to the recirculation loop in such a way that nitrogen is supplied to the recirculation loop.

18. The plant as claimed in claim 17, wherein the reformer includes a primary reformer and a secondary reformer for converting a hydrocarbon into hydrogen, wherein the primary reformer has a hydrogen side and a burner side, wherein the burner side is the combustion apparatus, wherein hydrocarbon is burned with air in the burner side of the primary reformer, wherein the burner side of the primary reformer is connected to a second carbon dioxide separator.

19. The plant as claimed in claim 18, wherein the second carbon dioxide separator is connected to the recirculation loop in such a way that nitrogen is supplied to the recirculation loop via the secondary reformer.

20. The plant as claimed in claim 17, wherein the combustion apparatus is a steam generation apparatus.

21. The plant as claimed in claim 20 wherein the second carbon dioxide separator is connected to the recirculation loop in such a way that nitrogen is supplied to the recirculation loop via the autothermal reformer.

22. The plant as claimed in claim 17, wherein the reformer is an autothermal reformer.

23. The plant as claimed in claim 17, wherein the second carbon dioxide separator is an ammonia-water scrubber.

24. The plant as claimed in claim 17, wherein the plant serves for the synthesis of ammonia and for the further synthesis of urea from the ammonia produced, wherein the plant includes a urea synthesis apparatus for the synthesis of urea from ammonia and carbon dioxide, wherein, for the separated carbon dioxide, the first carbon dioxide separator is connected to the urea synthesis apparatus, wherein the ammonia separator is connected to the urea synthesis apparatus in an ammonia conducting manner.

25. The plant as claimed in claim 17, wherein a dust extraction apparatus is arranged between the combustion apparatus and the second carbon dioxide separator.

26. The plant as claimed in claim 17, wherein the further hydrogen source and the second carbon dioxide separator are connected to the recirculation loop in such a way that the hydrogen stream from the further hydrogen source is first combined with the nitrogen stream from the second carbon dioxide separator and the mixture is then conveyed through a first compressor and thereafter conveyed through a methanator and then supplied to the recirculation loop.

27. The plant as claimed in claim 17, wherein the combustion apparatus is connected to the reformer.

28. The plant as claimed in claim 17, wherein a dust extraction apparatus is arranged between the combustion apparatus and the reformer.

29. The plant as claimed in claim 17, wherein a compressor is arranged between the combustion apparatus and the reformer.

30. The plant as claimed in claim 17 and for the further synthesis of urea from the ammonia produced, wherein the plant includes a urea synthesis apparatus for the synthesis of urea from ammonia and carbon dioxide, wherein the ammonia separator is connected to the urea synthesis apparatus in an ammonia conducting manner, wherein the second carbon dioxide separator is connected to the urea synthesis apparatus in such a way that carbon dioxide is supplied to the urea synthesis apparatus.

31. A process for expanding a capacity of an existing plant to include a further hydrogen source, wherein the further hydrogen source is connected to a recirculation loop in such a way that hydrogen is supplied to the recirculation loop, wherein a burner side of a primary reformer is connected to a secondary reformer.

32. A process for expanding a capacity of an existing plant, comprising:
expanding the capacity of the existing plant to include a further hydrogen source and a second carbon dioxide separator;
wherein the further hydrogen source is connected to a recirculation loop in such a way that hydrogen is supplied to the recirculation loop;
wherein a burner side of a primary reformer is connected to a second carbon dioxide separator;
wherein the second carbon dioxide separator is connected to the recirculation loop in such a way that nitrogen is supplied to the recirculation loop;
wherein the second carbon dioxide separator is connected to a urea synthesis apparatus in such a way that carbon dioxide is supplied to the urea synthesis apparatus.
